(19) United States
(12) Patent Application Publication
(10) Pub. No.: US 2019/0021845 A1
(43) Pub. Date: Jan. 24, 2019

(54) Cortical loop fixation system for ligament and bone reconstruction

(71) Applicant: Steven C. Chudik, Western Springs, IL (US)

(72) Inventor: Steven C. Chudik, Western Springs, IL (US)

(21) Appl. No.: 15/731,719

(22) Filed: Jul. 24, 2017

(51) Int. Cl. A61F 2/08

(52) U.S. Cl. CPC A61F 2/0811; A61F 2002/0852

(57) ABSTRACT

A system is disclosed for repairing and reconstructing an injured intra-articular, extra-articular ligament or tendon to a bone. The system includes realigning the axis of a bone, and further includes fixing fragments of a bone together. Novel devices, instruments and methods are disclosed, including a cortical loop for engaging a bundle of soft-tissue and capturing the bundle against a bone surface, an anchor for stabilizing a fractured bone, a guide for directing an orthopaedic blade, and a hinged external jig arranged to position and guide the blade. Methods are disclosed for connecting soft-tissue to a bone and for bone alignment.

### CLAIM FOR PRIORITY

This nonprovisional application is a division of the same inventor's nonprovisional application Ser. No. 13/998,567 filed in the United States Patent and Trademark Office on Nov. 12, 2013. Applicant claims, under 35 U.S.C. § 120, the priority benefit of the filing date of application Ser. No. 13/998,567 and, under 35 U.S.C. § 119(e), the priority benefit of the filing date of Nov. 19, 2012 of the same inventor's U.S. provisional Application No. 61/796,662 upon which Ser. No. 13/998,567 is based. The entire contents of each of these applications is hereby incorporated herein by reference.

Except for the provisional and nonprovisional applications just referred to, there are no patent applications related to this one. Neither this application nor the provisional and nonprovisional applications just referred to are subject to any federally sponsored research or development or to any joint research agreement.

### BACKGROUND OF THE INVENTION

The present invention relates to repairing and reconstructing injured ligaments and tendons. More particularly, it relates to novel devices, instruments and methods for repairing and reconstructing an injured intra-articular, extra-articular ligament or a tendon to a bone. It also relates to novel implants, instruments and methods for realigning the axis of a bone and for fixing fragments of a bone together.

Orthopaedic surgeons frequently perform reconstructive and reparative surgery for injured ligaments and tendons of the musculoskeletal system. When a patient traumatically injures a ligament in a joint, he may suffer from instability of that joint and require surgery to restore the function of the ligament and of the joint. Many ligament injuries cannot be directly repaired but rather require reconstructive surgery to make a new ligament by replacing the injured ligament with tendon graft. Likewise, when a patient traumatically ruptures a tendon of a muscle, he requires surgery to repair the tendon in order to restore the function of the muscle. Both the reconstruction of ligaments and repair of tendons involve mechanically connecting a soft-tissue tendon to the bone until the tendon can biologically reattach to the bone.

Orthopaedic surgeons also perform surgery to realign bones for patients who suffer from malalignment of bones and joints due to developmental and acquired disorders. The surgery, known as osteotomy, entails cutting a bone and realigning it along the cut, osteotomy, to change the alignment of the bone and adjacent joints.

Orthopaedic surgeons also perform surgery to repair fractures of bones, reconnecting the broken members so that they can heal in proper relationship. Repairing fractures of bones typically involves mechanically connecting the separated bone fragments, often with a properly sized and shaped bone fracture repair plate and screws.

### BRIEF SUMMARY OF THE INVENTION

The present invention discloses a new method, instruments and implants for reconstructing or replacing a damaged ligament of a joint or repairing a torn tendon by reattaching the bundle of soft-tissue directly to the cortical surface of a bone. The present invention also discloses a new manner of realigning a bone by performing an osteotomy and altering the relative position of the bone fragments. A novel implant utilizing an adjustable length bone fixation plate for repairing a fracture of a bone is also disclosed.

Accordingly, an object of this invention is to provide an improved means to restore a damaged ligament or tendon attached to a bone, thereby better restoring the normal anatomy of the joint and its normal structural relationships.

Another object of this invention is to better restore the anatomy of a joint by attaching a soft-tissue graft to the cortical surface of the ligament attachment site on the bone.

Another object of this invention is to avoid the surgical morbidity associated with drilling a large tunnel in a bone to reattach a ligament graft or torn tendon.

Another object of this invention is to avoid destruction of bone, creation of bone drilling debris, late tunnel widening, bone deficiency and similar drawbacks which complicate surgery.

Another object of this invention is to improve the functional outcomes of surgery because of an improved anatomic positioning of the graft and decreased surgical morbidity.

Another object of this invention is to provide for an osteotomy in a bone along a plane with a controlling relationship to an adjacent joint and its direction of movement.

Another object of this invention is to maximize the bony contact area across an osteotomy site to provide maximum stability and area of the bone healing surface.

Another object of this invention is to minimize changes to the longitudinal length of a bone following an osteotomy.

Another object of this invention is to minimize the distance between the joint and an osteotomy site while creating the osteotomy and realigning the bone at a position that maintains the relative length and function of the ligaments and tendons surrounding the joint.

Other objects and advantages of this invention will be apparent to orthopaedic surgeons and other persons who are skilled in the art of ligament and tendon repair and reconstruction, osteotomy, and bone fixation, particularly after reviewing the following description of the preferred embodiments of the present invention and the accompanying drawings.

### BRIEF DESCRIPTION OF THE DRAWINGS

FIG. 1A is a perspective view of a joint with two bones and an undamaged ligament connecting the bones at their bone attachment sites.

FIG. 1B is a perspective view of the joint in FIG. 1A illustrating the ligament in a damaged condition.

FIG. 1C is a perspective view of a joint similar to the joint in FIG. 1B with a ligament graft replacing the damaged ligament and fixed to the two bones within either a blind ended or complete bony tunnel in accordance with traditional techniques prior to the present invention.

FIGS. 2A, 2B, 2C and 2D are perspective views of the joint in FIG. 1B with alternative forms of a ligament graft replacing the damaged ligament and fixed to the two bones at the cortical surfaces of the bone attachment sites in accordance with the present invention.

FIGS. 3A, 3B, and 3C are perspective views of the joint in FIG. 1B with a folded ligament graft replacing the damaged ligament and fixed to the two bones at the cortical surfaces of the bone attachment sites in accordance with the present invention.

FIGS. 4A, 4B, 4C, 4D, and 4E are perspective views of the joint in FIG. 1B with alternative forms of a ligament graft replacing the damaged ligament and fixed to the two bones at the cortical surfaces of the bone attachment sites in accordance with the present invention.

FIGS. 5A, 5B, and 5C are perspective views of the joint in FIG. 1A with alternative forms of a folded ligament graft replacing the damaged ligament and fixed to the two bones at the cortical surfaces of the bone attachment sites in accordance with the present invention.

FIG. 6 is a perspective view of the joint in FIG. 1B with a graft augmented by a mechanical bridging material replacing the damaged ligament and fixed to the two bones at the cortical surfaces of the bone attachment sites in accordance with the present invention. Ideally these augmenting mechanical bridges anatomically follow the course of the native ligament and its bundles, attached to the bones at the native ligament attachment points.

FIGS. 7A and 7B are perspective views of the joint in FIG. 1B with alternative forms of a graft augmented by a fixation device which captures the free ends of the graft replacing the damaged ligament and fixed to the two bones at the cortical surfaces of the bone attachment sites in accordance with the present invention.

FIGS. 8A, 8B, 8C, 8D, and 8E are perspective views of the joint in FIG. 1B with alternative forms of a graft replacing the damaged ligament and fixed to the bones at the cortical surfaces of the bone attachment sites in accordance with the present invention.

FIG. 9 is a perspective view of a tendon attached to the cortical surface of a bone in accordance with the present invention.

FIG. 10 is a perspective view of a novel curved bone cutting guide which is utilized in the invention described herein.

FIG. 11A is a perspective view of the bone cutting guide shown in FIG. 10 aligned on a bone using a novel hinged external jig arranged across a joint for determining the plane of terminal flexion and extension of the joint in which the bone is a member.

FIG. 11B is a perspective view of the hinged external jig shown in FIG. 11A.

FIG. 12 is a perspective view of a bone being cut utilizing the curved bone cutting guide shown in FIG. 10.

FIG. 13 is a perspective view of a bone which has been cut using the curved bone cutting guide of FIG. 10 disposed for realignment in accordance with the present invention.

FIG. 14 is a perspective view of a fractured bone repaired with a traditional plate and screws.

FIG. 15 is a perspective view of a fractured bone with a novel adjustable-length compression fracture plate placed across the fracture in the bone in accordance with the present invention.

FIG. 16 is a perspective view of a number of intercalary segments having various cross sectional configurations which may be used with the fracture plate shown in FIG. 15.

FIG. 17A is a perspective view of a novel double locking screw arranged for engagement with the fracture plate of FIG. 15

FIG. 17B is a perspective view of a conventional screw arranged for engagement with the fracture plate of FIG. 15.

FIG. 18 is a sectional view of the fracture plate in FIG. 15 applied to the bone taken along the line 1-1 in FIG. 15.

FIG. 19 is a sectional view taken along the same line 1-1 in FIG. 18 of the fracture plate in FIG. 15 applied to a bone and utilizing a double locking screw as shown in FIG. 17A.

### DETAILED DESCRIPTION OF THE INVENTION

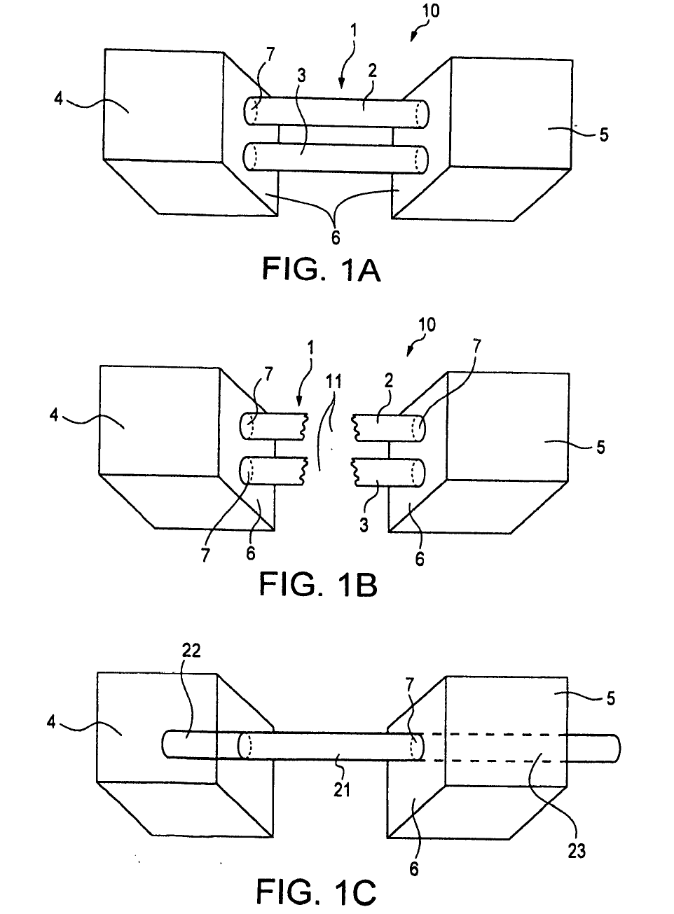

An undamaged joint 10 is illustrated in FIG. 1A which includes a first bone 4 and a second bone 5 connected by a ligament 1. Such ligaments 1 may consist of multiple separate bundles such as ligament bundles 2,3 which connect to the bone attachment sites 7 on the cortical surfaces 6 on the bones 4,5.

Ligaments of a joint can be torn from injury, as shown in FIG. 1B. Primary repair of the torn ligaments 11 themselves is usually unsuccessful and orthopaedic surgeons typically replace them by drilling either a blind ended tunnel 22 or a tunnel completely through the bone such as tunnel 23 at the ligament attachment sites 7 and place a biologic soft-tissue graft such as the graft 21 in the tunnels 22, 23 to replace the spanning ligament 1. That construction is shown at FIG. 1C.

Drilling tunnels in a bone creates injury, does not allow for anatomic attachment of a graft to the cortical surface, and sometimes leads to other surgically related complications.

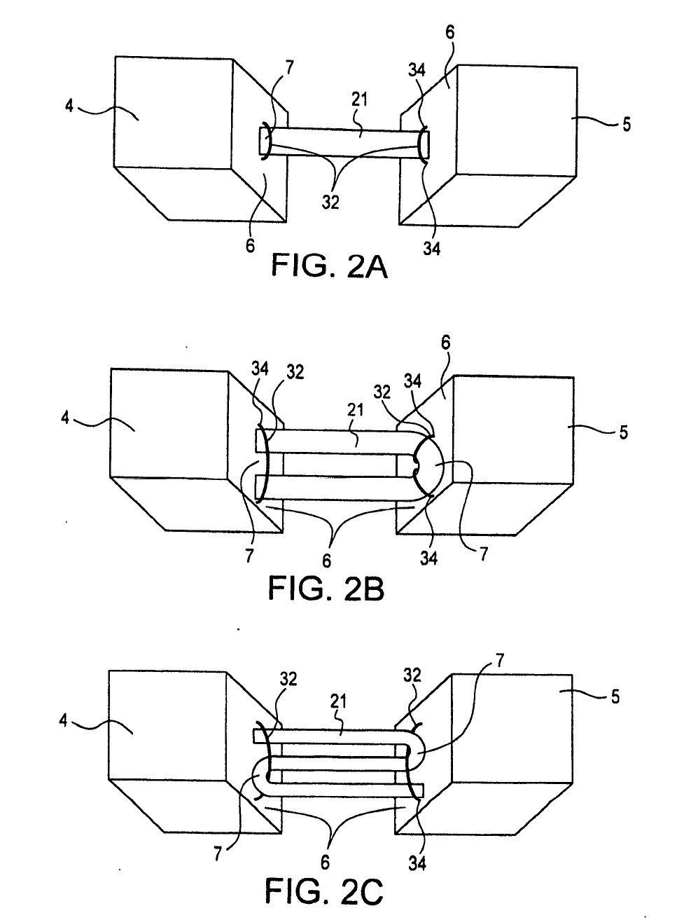

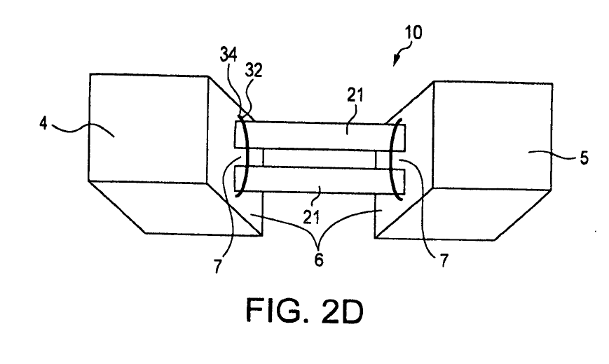

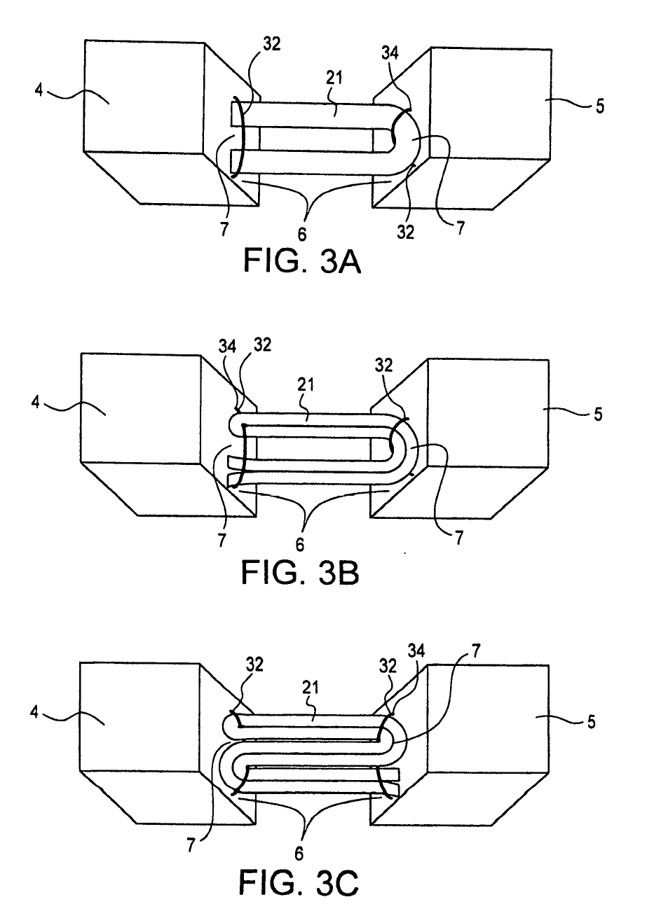

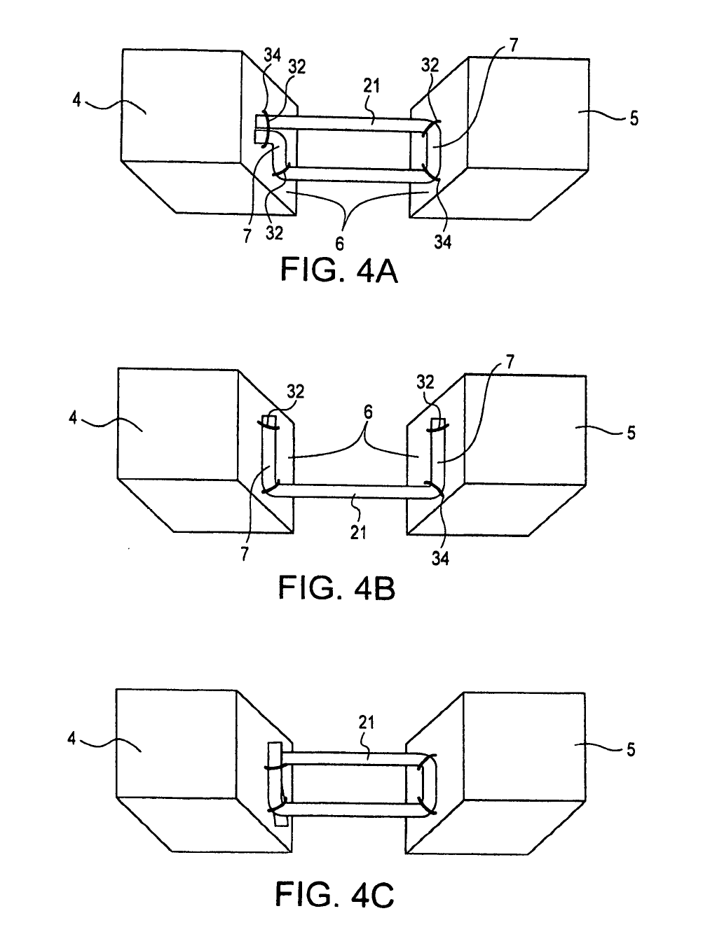

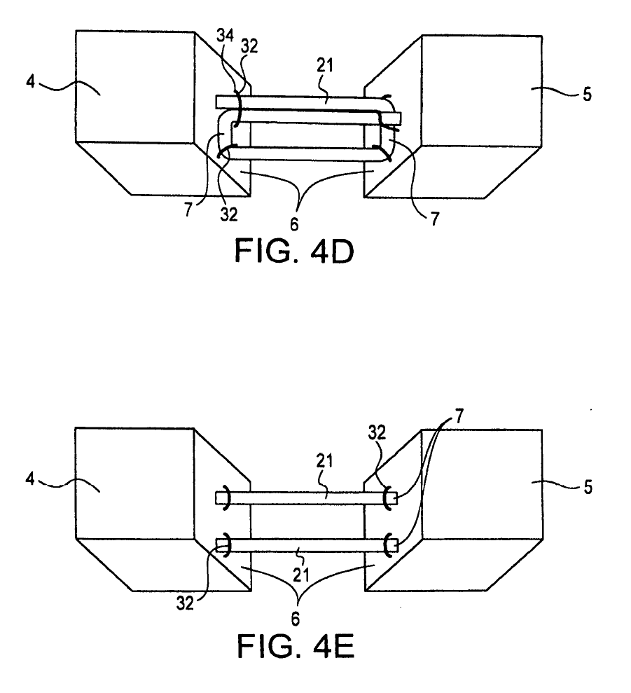

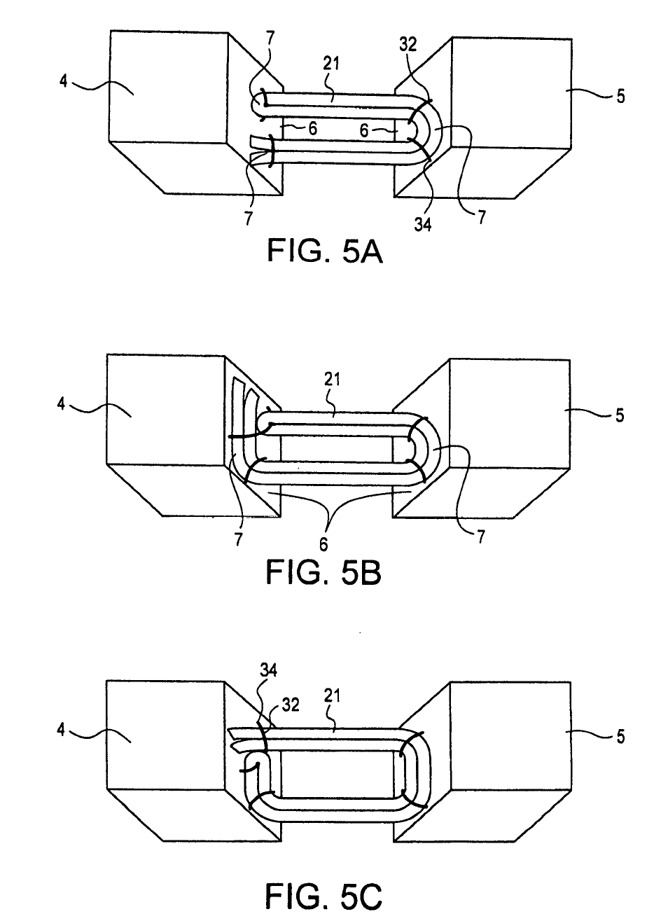

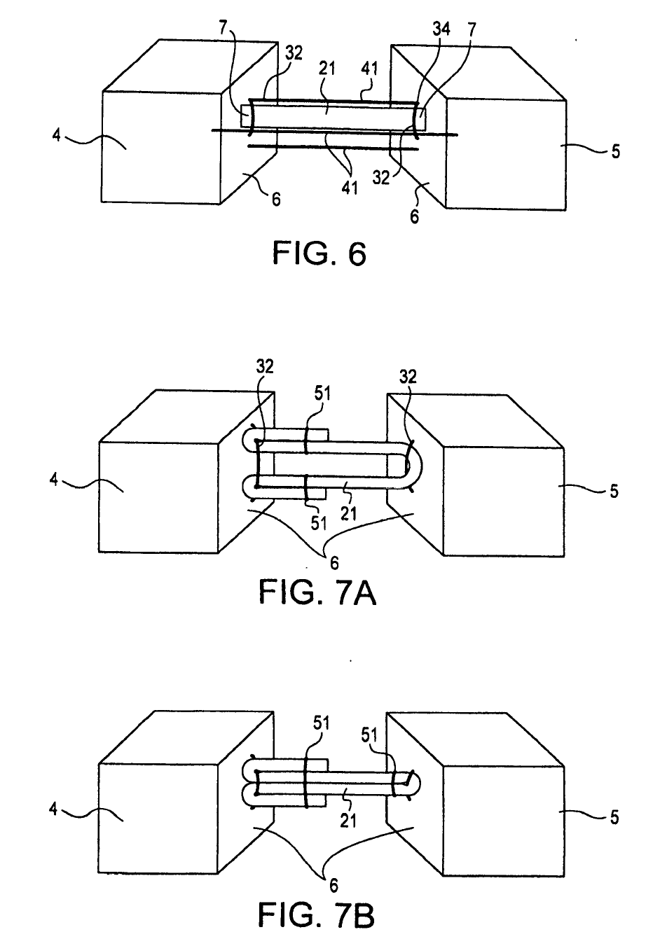

The novel method, technique and implant of the present invention reconstructs a ligament such as ligament 1 by connecting a biologic soft-tissue graft 21 to the cortical surfaces 6 at the ligament attachment sites 7 located on the bones 4 and 5 of a joint 10. Various forms which the graft 21 may take are shown in FIGS. 2 through 9.

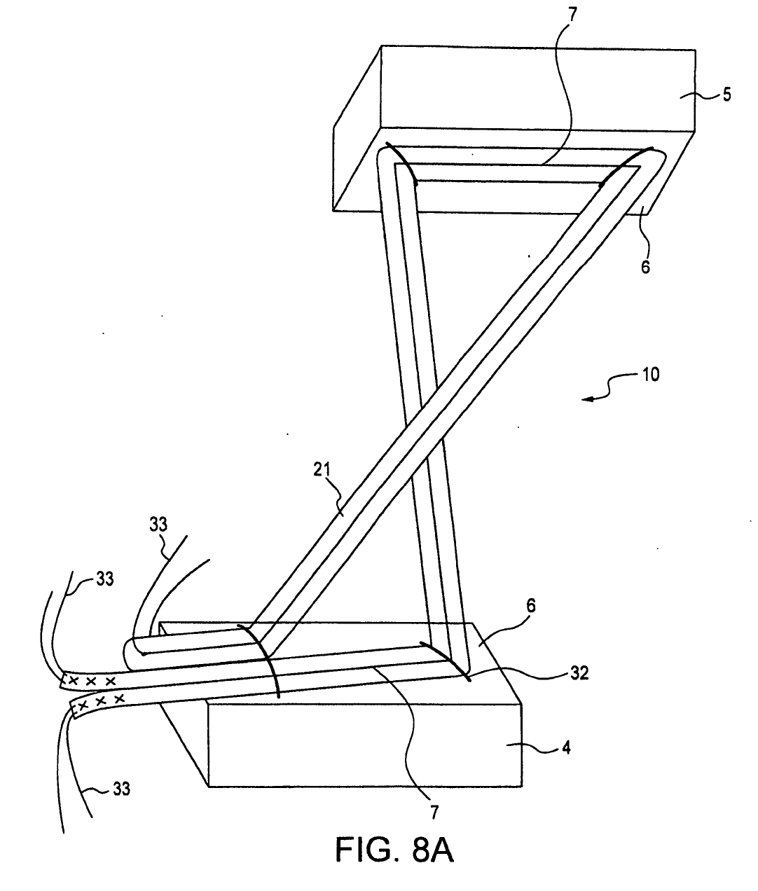

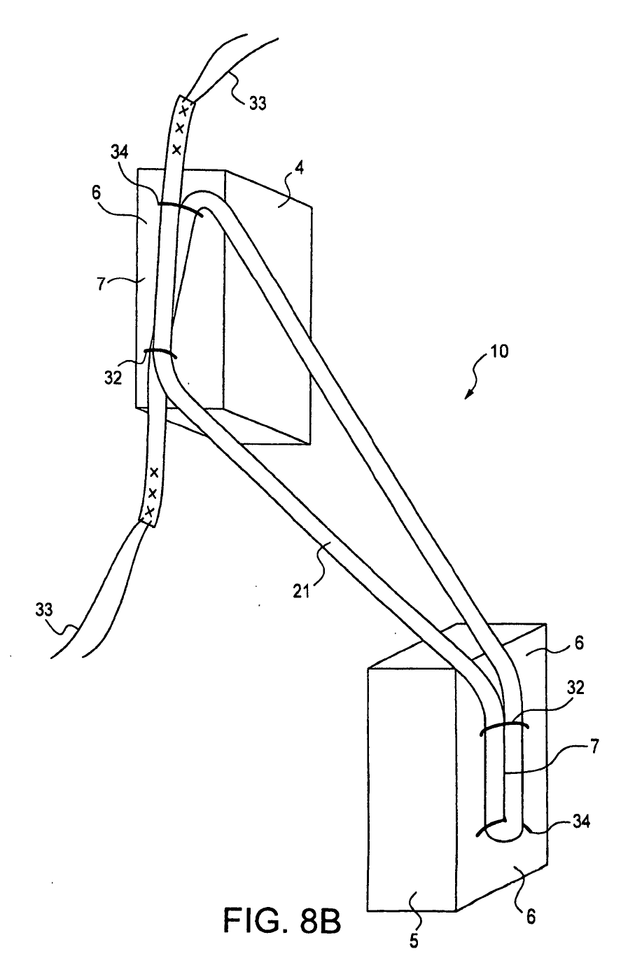

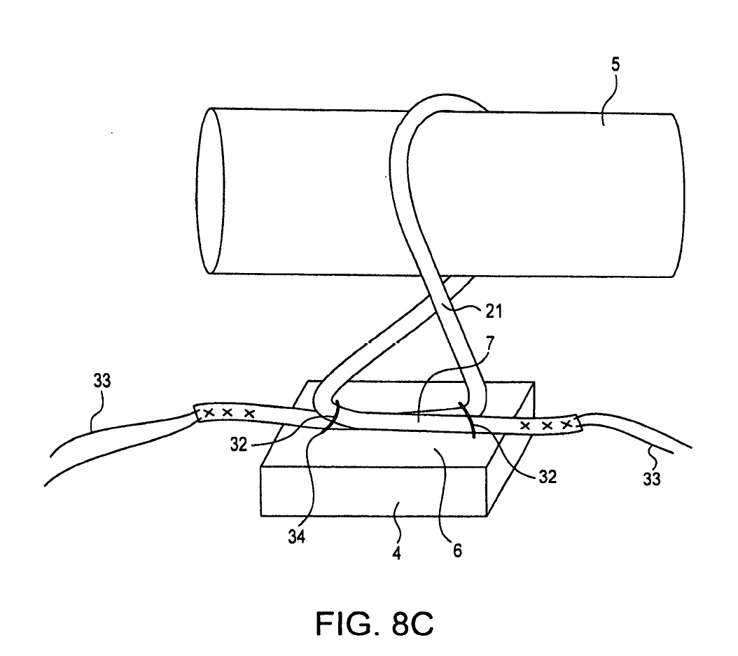

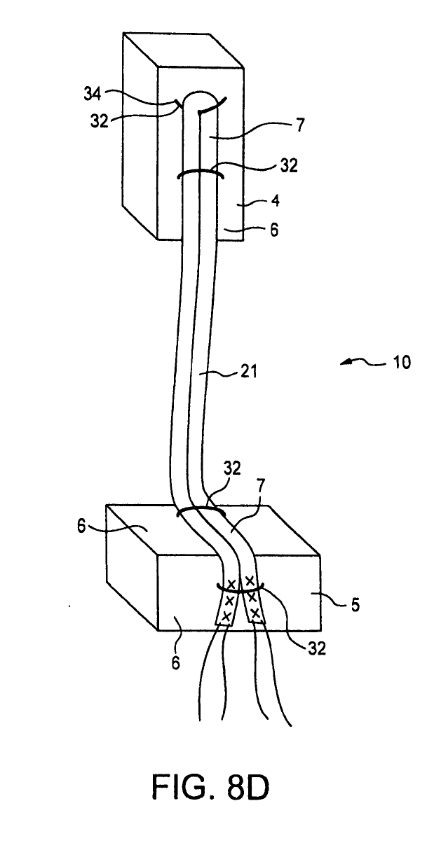

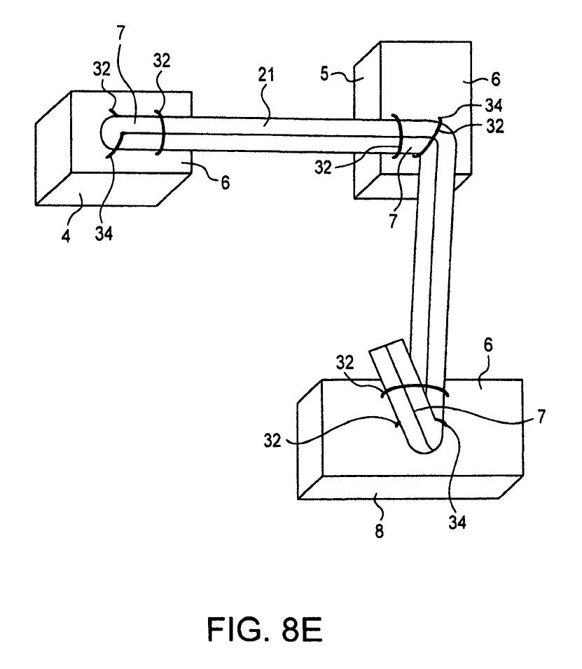

At the time of ligament reconstructive surgery, with or without arthroscopic, fluoroscopic, robotic, or computer navigational assistance, cortical loops 32 are placed along a cortical surface 6 at a ligament bundle attachment site 7 capturing the soft-tissue graft 21. The cortical surface of the bone may be prepared to stimulate a healing response at that site. The cortical loops 32, which may be called fixation devices, may be of a fixed length or adjustable length and composed of permanent, biologic, composite, or resorbable biocompatible material, and they can be of varying diameter or width. Each loop 32 engages the bone at two separate sites 34 with intervening cortical bone surface between. Multiple cortical loop fixation devices 32 and multiple biologic soft-tissue grafts 21 can be used to in a multitude of configurations to restore the multiple bundle 2,3 anatomy of the original ligament 1 across the bones 4, 5, 8 proximate to a joint 10. Additional mechanical reinforcements 41 can be used to bridge the bones 4,5 to mechanically reinforce graft 21 until it heals to bones 4 and 5, and mechanical graft locks 51 can be added to fix loose ends of the graft back upon itself to create a closed loop as shown in FIGS. 7A and 7B. Grasping suture or other mechanical devices 33 placed at either or both of the loop and free ends of a soft-tissue graft or the ends of the graft themselves can be anchored to the bone 4 at a distant site to augment the overall fixation strength of ligament reconstruction construct (FIG. 8A)

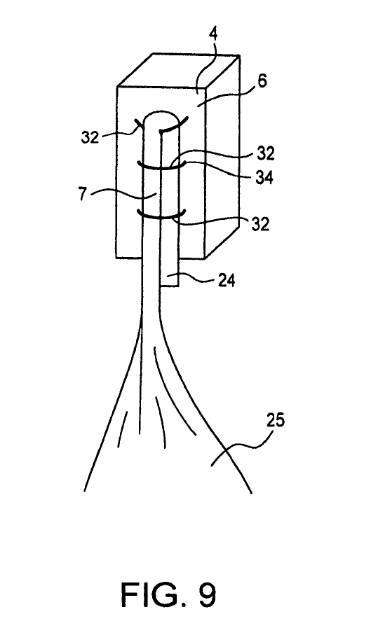

Cortical fixation loops 32 can also be used in multiple configurations to fix torn tendons 24 of muscles 25 back to a cortical surface 6 of a bone 4, as shown in FIG. 9, for example.

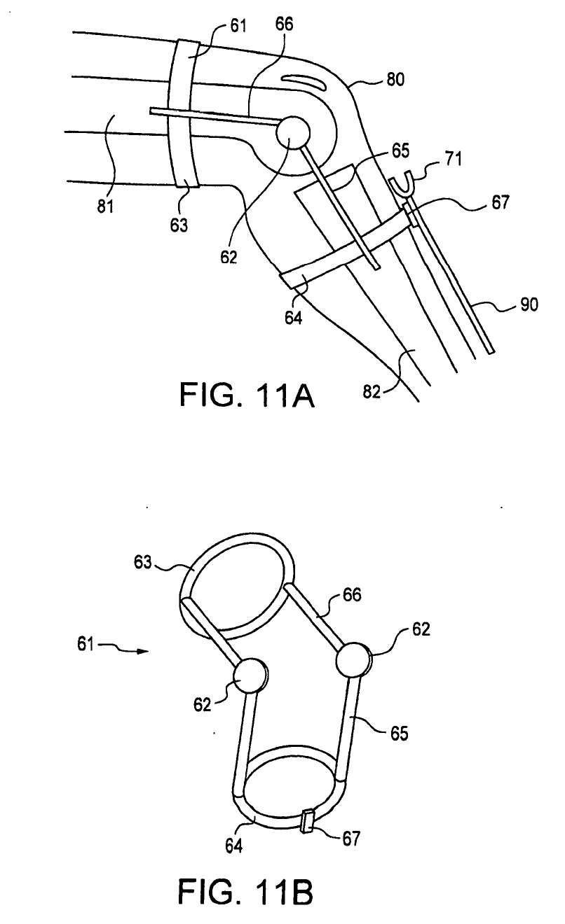

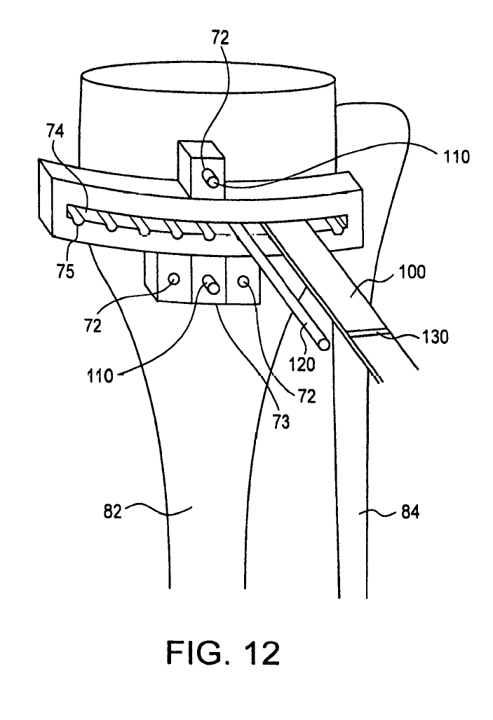

Bone realignment procedures are also sometimes needed in conjunction with ligament reconstructive procedures. In the present invention, the following novel bone realignment procedures may be utilized. A hinged external jig 61 and bone cutting guide 71 may be created using patient-specific three-dimensional anatomic data from preoperative imaging modalities and computer software, including but not limited to computed tomographic scans, magnetic resonance imagining and plain radiography. The hinged external jig 61 can be applied to a joint 80 with fasteners 63, 64 that help position the jig 61 along bones 81, 82 of the joint 80, thereby positioning the hinge 62 with its two connecting arm 65, 66 at the center of rotation of the joint 80 as shown in FIG. 11A. Fasteners 63, 64 may be applied to the bones 81, 82 but surrounding soft-tissue or similar bone fixation devices around the joint 80 may be used instead. The hinged external fixation jig 61 may be unilateral (single hinge 62 and arms 65, 66) or bilateral (two hinges 62 and two sets of arms 65, 66).

The bone cutting guide 71 is removably attached to the hinged external jig 61 through the jig connector 67. The joint 80 is moved to demonstrate the plane of bending motion, as shown in FIG. 11A. Also, the jig connector 67 allows cranial-caudal and rotational adjustment of the cutting guide position. A longitudinal alignment rod 90 can be removably attached to the cutting guide 71 to help adjust the longitudinal alignment in the coronal and sagittal planes. When the cutting guide is arranged in proper position, i.e., height, rotation, slope (tilt), and perpendicular to the plane of flexion of the joint 80, it is fixed to the bone 82 with bone fixation devices 110 and the external hinged jig 61 is removed as shown in FIG. 12.

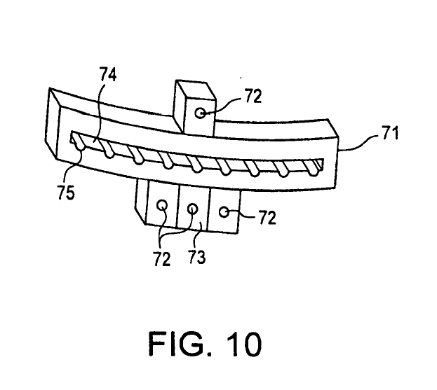

The cutting guide 71 includes cutting holes 75, a slot 74, stabilizing arms or tabs 73 and holes 72 for bone fixation devices 110 (see FIGS. 10 and 12). With the cutting guide 71 fixed in position on bone 82, pins of fixed length 120 may be inserted into the bone through holes 75 in the cutting guide 71. Each pin can possess a slightly larger cutting diameter at its leading tip which allows the pin to cut through a first cortex of a bone 82 and pass with relatively little tactile resistance through the central cancellous portion of bone 82 until pin 120 reaches the second cortex and meets resistance again. By placing several fixed length pins 120 in this fashion, the thickness of the bone 82 and the required depth of the cut can be determined. Then a cutting blade 100 can be advanced through cutting slot 74 to a depth which matches depth mark 130 on the cutting blade 100 corresponding to the length of pins 120 until the mark 130 on the blade 100 reaches the trailing end of the fixed length pins 120 as shown in FIG. 12.

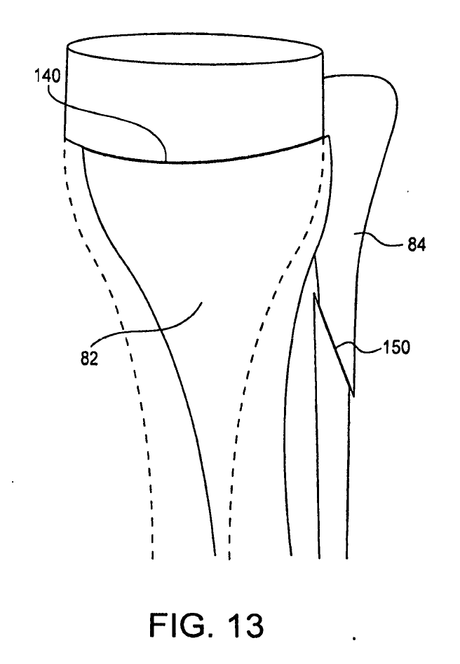

When the bone 82 is cut, and the cutting guide 71 is removed, any adjacent supporting bone such as bone 84 is also cut, and bones 82 and 84 are realigned along their respective osteotomies 140, 150. There they are fixed in position as shown in FIG. 13.

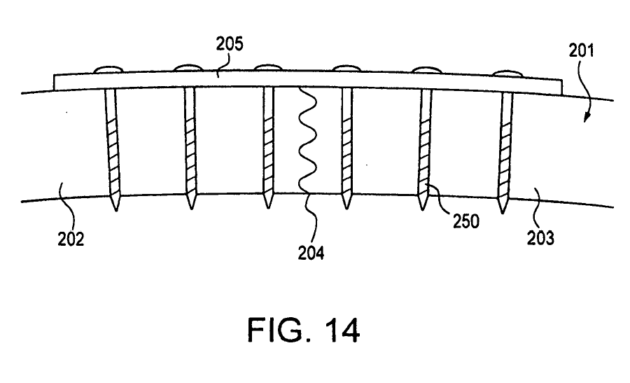

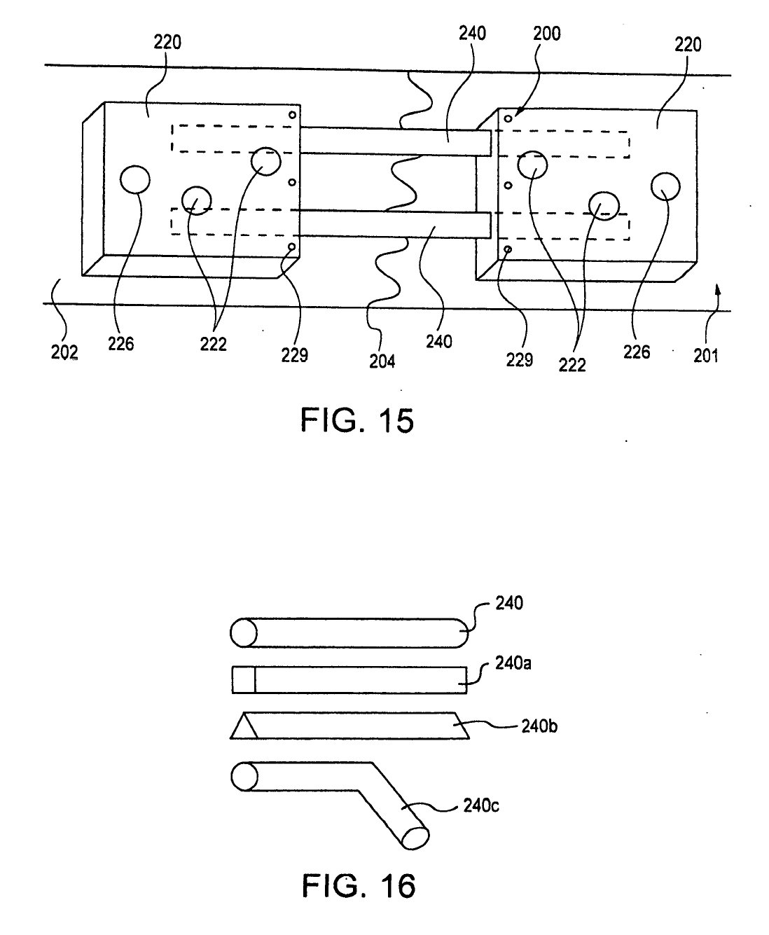

The present invention includes fixation of bone 201 fragments 202, 203 from osteotomies 140 or fracture 204. After percutaneous or open exposure of a bone 201 with a fracture 204, a conventional plate 205 and screws 250 can be applied as shown in FIG. 14. In the present invention, as shown in FIG. 15, after a similar exposure novel modular fracture plate ends 220 are connected with single or multiple intercalary segments 240 to assemble the locking adjustable-length compression fracture plate 200. That plate is applied to the bone 201, centered over the fracture 204. Proper length and size intercalary segments 240 can be inserted into the intercalary segment channels 221 of the fracture plate ends 220. Modular fracture plate ends 220, intercalary segments 240, and locking screws 230 can be made in various sizes in order to fix different sized bones. Intercalary segments 240 can also be made in different cross sectional shapes, stiffness, longitudinal shape, bending properties, materials (including radiolucent, biologic, resorbable) and lengths such as 240a, 240b and 240c (see FIG. 16).

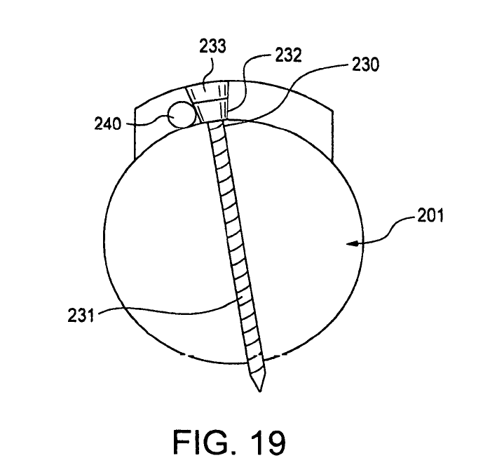

After the adjustable length plate 200 is applied to the reduced fracture 204, locking screws 230 or non-locking screws 250 are inserted through the regular screw single-locking and non-locking locking holes 226 in the fracture plate ends 220 to connect the fracture plate ends 220 to the bone 201. Optionally, a locking compression-reduction clamp (not shown) can be used to grasp each of the fracture plate ends 220 by their respective compression device attachment points 229 and apply compression and reduction forces across fracture 204. Then, with the compression clamp in place, double locking screws 230 can be inserted into the combination screw locking and intercalary segment locking holes 222 for locking engagement between the fracture plate ends 220 and the intercalary segment 240 as shown in FIGS. 18 and 19.

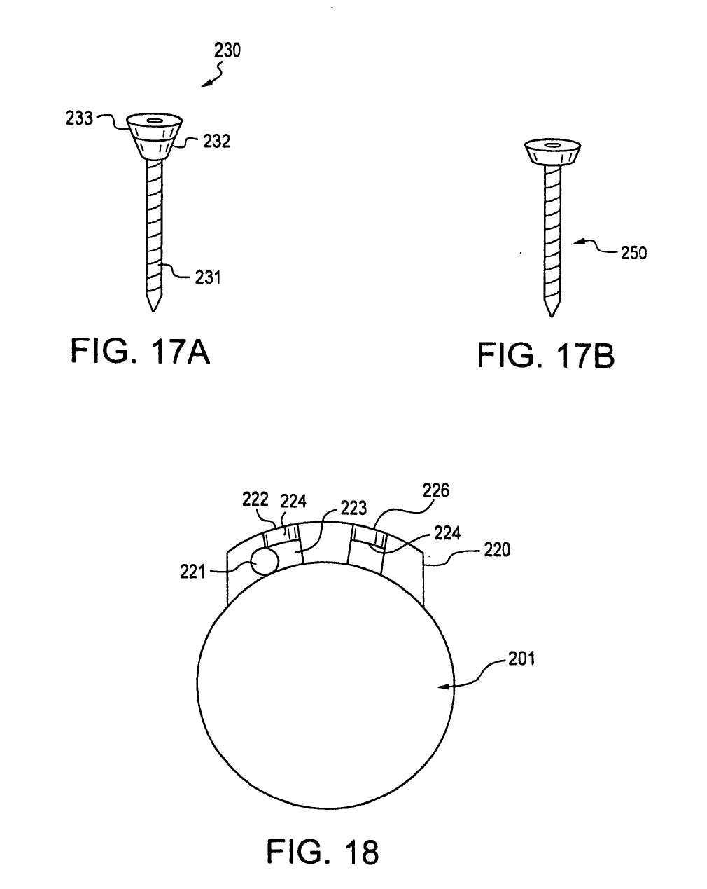

Locking engagement connects the double locking screw 230 at a fixed position, depth and angle relative to the fracture plate ends 220 and also compresses the intercalary segment 240 within the intercalary segment channel 221 of the fracture plate ends 220 so as to prevent any longitudinal or rotational movement between the fracture plate ends 220 and the intercalary segment 240 along the axis of the intercalary segment 240. This locking engagement can be reversed with removal of the locking screw 230. The double locking screws 230 can have combined or separate plate and intercalary segment engaging sections 233, 232 and a threaded bone engaging section 231 as shown in FIG. 17A. The plate and intercalary segment engaging sections 233, 232 of the double locking screw 230 reversibly engage plate engaging section 224 and intercalary segment-plate engaging section 223. These interacting sections of the holes 222 and screws 230 may refer to physically separate segments or overlapping segments, or to the same segments of the holes 222 and screw 230, depending upon which engaging mechanisms are incorporated. The intercalary channels 221 may be completely enclosed or partially enclosed within the fracture plate ends 220.

From the foregoing it will be evident that, although particular forms of the present invention have been illustrated and described, nevertheless various modifications can be made without departing from the true spirit and scope of the invention. Accordingly, no limitations are intended by the foregoing description and the accompanying drawings, and the true spirit and scope of the invention are intended to be expressed in the following claims.

I claim:

1. A method of connecting soft tissue to a bone comprising the steps of
placing loops having adjustable length at sites on the cortical surface of the bone,
placing the soft tissue on the cortical surface and through the loops, and
tensioning the soft tissue and the loops to form contact of the soft tissue on the cortical surface.

2. The method of claim 1 in which the soft tissue is a ligament.

3. The method of claim 1 in which the soft tissue is a tendon.

4. The method of claim 1 in which the soft tissue is a graft.

5. The method of claim 1 which includes a step of preparing the cortical surface to receive the soft tissue.

6. The method of claim 1 in which the soft tissue is a folded graft.

7. The method of claim 1 in which the soft tissue is an unfolded graft.

8. The method of claim 1 in which distal ends of the loops are placed on the cortical surface of the bone at separate sites.

9. The method of claim 1 which includes the step of supporting the soft tissue with a mechanical reinforcement.

10. The method of claim 1 which includes connecting free ends of the soft tissue to the bone.

11. The method of claim 1 which includes connecting a free end of the soft tissue to a second site spaced apart from the loops.
